SEQUENCE LISTING (16 sequences)

SEQ ID NO: 1 - protein, 9 aa, synthetic construct
FLLDGSANV

SEQ ID NO: 2 - protein, 9 aa, synthetic construct
FLLDGSEGV

SEQ ID NO: 3 - protein, 9 aa, synthetic construct
FLLDGSINF

SEQ ID NO: 4 - protein, 24 aa, synthetic construct
MTEYKLVVVGADGVGKSALTIQLI

SEQ ID NO: 5 - protein, 24 aa, synthetic construct
MTEYKLVVVGAVGVGKSALTIQLI

SEQ ID NO: 6 - protein, 24 aa, synthetic construct
MTEYKLVVVGARGVGKSALTIQLI

SEQ ID NO: 7 - protein, 24 aa, synthetic construct
MTEYKLVVVGACGVGKSALTIQLI

SEQ ID NO: 8 - protein, 24 aa, synthetic construct
MTEYKLVVVGASGVGKSALTIQLI

SEQ ID NO: 9 - protein, 39 aa, synthetic construct
LSGTPEVHSNKRDIIFLLDGSANVGKTNFPYVRDFVMNL

SEQ ID NO: 10 - protein, 39 aa, synthetic construct
HNGAPAPVSGEKDVVFLLDGSEGVRSGFPLLKEFVQRVV

SEQ ID NO: 11 - protein, 39 aa, synthetic construct
TPPPSRPEKKKADIVFLLDGSINFRRDSFQEVLRFVSEI

SEQ ID NO: 12 - protein, 24 aa, synthetic construct
MTEYKLVVVGADGVGKSALTIQLI

SEQ ID NO: 13 - protein, 24 aa, synthetic construct
MTEYKLVVVGAVGVGKSALTIQLI

SEQ ID NO: 14 - protein, 24 aa, synthetic construct
MTEYKLVVVGARGVGKSALTIQLI

SEQ ID NO: 15 - protein, 24 aa, synthetic construct
MTEYKLVVVGACGVGKSALTIQLI

SEQ ID NO: 16 - protein, 24 aa, synthetic construct
MTEYKLVVVGASGVGKSALTIQLI

(19) United States
(12) Patent Application Publication
(10) Pub. No.: US 2024/0325448 A1
(43) Pub. Date: Oct. 3, 2024

(54) COLD ATMOPSHERIC PLASMA TREATED PAN-CANCER EPITOPE PEPTIDE WITHIN THE COLLAGEN TYPE VI A-3 (COL6A3) PROTEIN AS CANCER VACCINE

(71) Applicant: JEROME CANADY RESEARCH INSTITUTE FOR ADVANCED BIOLOGICAL AND TECHNOLOGICAL SCIENCES, Takoma Park, MD (US)

(72) Inventors: Jerome Canady, Takoma Park, MD (US); Saravana Murthy, Takoma Park, MD (US)

(21) Appl. No.: 18/702,095

(22) PCT Filed: Sep. 28, 2023
(86) PCT No.: PCT/US2023/033964

§ 371 Date: Apr. 17, 2024

Related U.S. Application Data
(60) Provisional application No. 63/411,586, filed on Sep. 29, 2022.

(51) Int. Cl. A61K 35/17; A61K 39/00; C07K 7/06; C12N 5/0783; C12N 13/00

(52) U.S. Cl. CPC A61K 35/17; A61K 39/4611; C07K 7/06; C12N 5/0636; C12N 13/00; C12N 2501/2307; C12N 2501/2315; C12N 2501/998; C12N 2502/30

(57) ABSTRACT

A method to oxidize pan-cancer epitopes of COL6A3 protein (peptide sequences 1. FLLDGSANV (SEQ ID NO: 1), 2. FLLDGSEGV (SEQ ID NO: 2) and 3. FLLDGSINF (SEQ ID NO: 3)) by cold atmospheric plasma treatment for developing solid tumor cancer vaccines.

### CROSS-REFERENCE TO RELATED APPLICATIONS

The present application claims the benefit of the filing date of U.S. Provisional Patent Application Ser. No. 63/411,586 filed by the present inventors on Sep. 29, 2022.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field Of The Invention

The present invention relates to cancer treatments, and more specifically, to cold atmospheric plasma treated peptides.

### Brief Description Of The Related Art

Immunotherapy is broadly termed for a number of different modalities that are all designed to use the body's own immune system to help fight cancer. The specifics of each therapy differ, but they all rely on a deep understanding of how the immune system is regulated and how that regulation can be modified to get the desired outcome. For the past several years it has been known that the immune system plays an important role in monitoring the formation of potentially cancerous cells. It has been reckoned that a diploid mammalian cell can undergo ˜20,000 DNA mutational events a day. However, the vast majority of these events are identified and repaired by specific DNA repair pathways. Those cells that do acquire a malignancy and are not repaired are usually removed by the body's immune surveillance system. This generally involves cell mediated immune mechanisms that rely on differentiating between self and non-self-antigens. Most often the DNA mutations are a single point mutation in a nucleotide that leads to a unique peptide (neoantigen) that may be recognized by the immune system as non-self these Tumor Associated Antigens (TAA) are presented on the surface of cells in conjunction with Major Histocompatibility (MHC) molecules. This TAA-MHC complex can then be recognized by a T-cell with an appropriate T-cell Antigen Receptor (TCR). However, in order for the T cell to become activated a complex mix of co-stimulatory signals are required. One example of these types of signals is the CD28, CD80/CD86 activation signal. CD80 and CD86 proteins are expressed on Antigen Presenting Cell (APC) and are required to engage their shared receptor, CD28, for the co-stimulation of CD4 T-cell to carry out its effective function.

Peptide vaccines are a type of vaccination approach that uses synthetic tumor-associated or specific peptides, or a combination of peptides, designed to elicit peptide-specific T-cells. These peptides are presented on the surface of human leukocyte antigen (HLA) molecules for recognition by T cell receptors of CD4+ and CD8+ T cells. Peptide vaccines have shown benefits in treating metastatic cancers since they lack significant toxicity associated with chemotherapy.

Many recent personalized cancer vaccine developments, include mRNA and protein fragments, or peptides. Investigational mRNA vaccines are manufactured for individuals based on the specific molecular features of their tumors. This approach elicits an immune response against abnormal proteins, or neoantigens, produced by cancer cells. Because these proteins are not found on normal cells, they may be more effective targets for cancer vaccines.

Several new developments offer promise for improving peptide vaccines, including the use of long peptides and optimization of adjuvants, including toll-like receptors. Peptide vaccines offer a promising strategy to elicit specific anti-tumor immune responses. Peptide vaccines have shown benefits in treating metastatic cancers since they lack significant toxicity associated with chemotherapy. Peptide-based subunit vaccines, including chemical and biosynthetic preparations of predicted or known specific tumor antigens, induce a robust immune response against the particular tumor antigen site. However, there is currently no consensus regarding the most optimal adjuvant to be used for a given peptide vaccine, and this could be a promising research area to further optimize and improve vaccine formulations.

Cold Atmospheric Plasma (CAP) treatment has been investigated as a potential method for enhancing the efficacy of peptide vaccines for cancer treatment. CAP can modify the structure of proteins, which can improve their mechanical properties and stability. The possible mechanisms that could contribute to the stabilization of proteins by CAP include oxidative damage, interaction with amino acids, Interfacial performance and modification of protein properties. CAP generate reactive oxygen and nitrogen species (RNOS) that can induce oxidative damage to proteins. This oxidative damage can lead to protein cross-linking and stabilization. CAP is also composed of other reactive species, such as radicals and excited atoms and molecules, that can interact with proteins. The specific reactive species involved in the interaction between cold plasma and proteins may vary depending on the specific experimental conditions and the type of protein being treated. CAP interact with amino acids present in proteins, leading to modifications in their structure and properties.

The use of CAP treated peptide vaccines could offer a more targeted and less toxic approach to cancer treatment. Several different systems and methods for performing Cold Atmospheric Plasma (CAP) treatment have been disclosed.

For example, U.S. Pat. No. 10,213,614 discloses a two-electrode system for CAP treatement. U.S. Pat. No. 9,999,462 and U.S. Pat. No. 10,023,858 each disclose a converter unit for using a traditional electrosurgical system with a single electrode CAP accessory to perform CAP treatment. WO 2018191265A1 disclosed an integrated electrosurgical generator and gas control module for performing CAP.

### SUMMARY OF THE INVENTION

In a preferred embodiment, the present invention is a method to oxidize pan-cancer epitopes of COL6A3 protein (peptide sequences FLLDGSANV (SEQ ID NO: 1), FLLDGSEGV (SEQ ID NO: 2) and FLLDGSINF (SEQ ID NO: 3) by cold atmospheric plasma treatment for developing solid tumor cancer vaccines. The method may comprise the steps of synthesizing and solubizing peptides FLLDGSANV (SEQ ID NO: 1), FLLDGSEGV (SEQ ID NO: 2) and FLLDGSINF (SEQ ID NO: 3) in an appropriate solvent such as PBS or DMSO; treating the synthesized peptides with cold atmospheric plasma (CAP); isolating Pan T cells from peripheral blood mononuclear cells (PBMC); cultivating Pan T cells in TexMACSTM Medium supplemented with IL-7 and IL-15; activating the Pan T cells with T Cell TransAct; transducing the Pan T cells with fluorescence protein construct using a lentiviral vector; directly applying CAP treated peptides to naïve T cell culture or to dendritic cell culture for 3 to 7 days; expanding T cells in TexMACS Medium supplemented with IL-7 and IL-15, T cells are split every 2-3 days; washing T cell cultures and remove cytokines; co-culturing a subset of T cells with target cells for 48 h; analyzing killing of target cells; and upon successful killing of target cells, isolate and store T cells until administered to patient. The isolating of Pan T cells from peripheral blood mononuclear cells (PBMC) may be performed using a commercially available Pan T cell Isolation Kit, human. The step of treating the synthesized peptides with cold atmospheric plasma (CAP) may comprise treating the synthesized peptides with cold atmospheric plasma at a power ranging from 1 v to 120 v, and time from 1 second to 7 minutes with 1 to 3 liters per minute of Helium.

In another preferred embodiment, the present invention uses cold atmospheric plasma-treated cancer epitope peptides within the KRAS protein as a PDAC vaccine. The cold atmospheric plasma treatment will increase the immunogenicity of the KRAS protein, making it a more effective target for cancer immunotherapy.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 shows a method for cold atmospheric plasma treated pan-cancer epitope peptide within the collagen type vi α-3 (col6a3) protein as cancer vaccine in accordance with a preferred embodiment of the present invention. FIG. 1 discloses SEQ ID NOS: 1-3, respectively, in order of appearance.

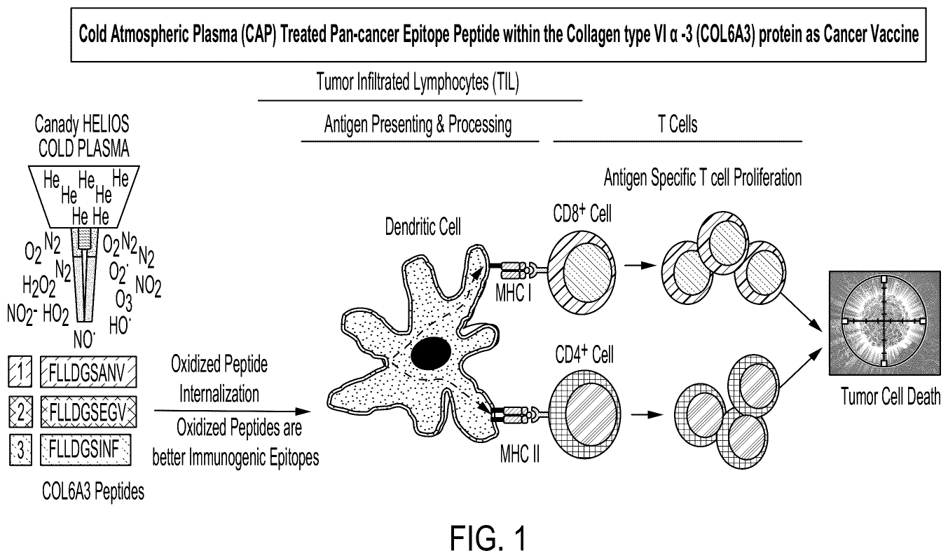

FIG. 2 shows a method for cold atmospheric plasma treated cancer epitope peptide within the KRAS protein as PDAC vaccine in accordance with another preferred embodiment of the present invention. FIG. 2 discloses SEQ ID NOS: 12-16, respectively, in order of appearance.

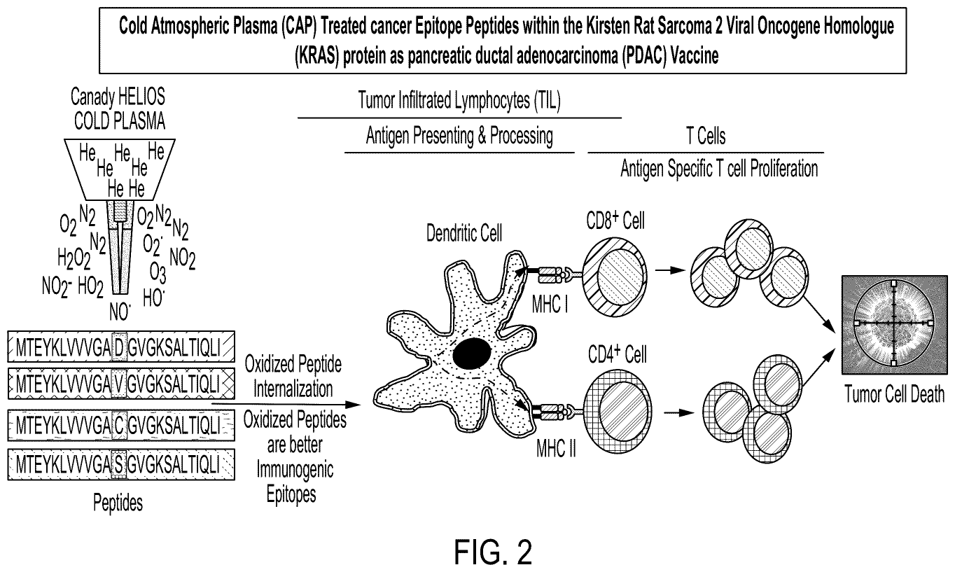

### DETAILED DESCRIPTION OF THE PREFERRED EMBODIMENTS

The tumor microenvironment (TME) is a complex network of cells and structures that surround and interact with cancer cells in a tumor. The TME is comprised of many different cell types including cancer cells a wide variety of immune cells and cells involved in structural support like stromal cells. In the TME, the cancer cells can manipulate and hijack normal cell function to better serve their purpose allowing cancer cells to survive and multiply. One major activity of cancer cells is evading the immune system and promoting an immunosuppressive TME. CAR-T cell therapy have range of limitations, including finding targets that are tumor specific, the immunosuppressive nature of the tumor microenvironment (TME), and tumor immune escape mechanisms. There have been several efforts to overcome the limitations of CAR-T therapy, one such is to identifying tumor-specific mutations and peptides derived thereof (neoepitopes), which are presented by human leukocyte antigen (HLA). Using mass spectrometry (MS) several peptide-HLA (pHLA) complex have been identified that are located in a particular immunological synapse. To specifically target the TME few approaches have been developed, such as to target fibroblast-associated protein (FAP), highly expressed in epithelial cancer with limited expression in normal tissues, however, targeting FAP showed bone marrow toxicity. Recent study showed the pan-cancer epitope derived from a tumor-specific splice variant of COL6A3 gene that is highly presented on tumor stroma across multiple solid cancers due to a tumor-specific alternative splicing event that rarely occurs outside the tumor microenvironment and this pHLA was identified analyzing 739 tumor and 673 normal tissue samples.

Cold atmospheric plasma treatment of cancer cells can oxidize proteins and induces cell death. These oxidized proteins are internalized by antigen presenting cell (APC) and these oxidized protein/peptides are presented to T cells an HLA-peptide complex. Oxidized proteins derived from COLD ATMOSPHERIC PLASMA treated apoptotic cells are better immunogenic epitopes that could induce a strong immune response against the tumor cells. The present invention is a method to oxidize pan-cancer epitopes of COL6A3 protein (peptide sequences 1. FLLDGSANV (SEQ ID NO: 1), 2. FLLDGSEGV (SEQ ID NO: 2) and 3. FLLDGSINF (SEQ ID NO: 3)) by COLD ATMOSPHERIC PLASMA treatment for developing solid tumor cancer vaccines.

The method includes the following steps:

1) Peptides, 1. FLLDGSANV (SEQ ID NO: 1), 2. FLLDGSEGV (SEQ ID NO: 2) and 3. FLLDGSINF (SEQ ID NO: 3) are synthesized and solubilized in an appropriate solvent such as PBS or DMSO.

2) The peptides are treated with cold atmospheric plasma (CAP) at power ranging from 1 v to 120 v, and time from 1 second to 7 minutes with 1 to 3 liters of He.

3) Pan T cells from peripheral blood mononuclear cells (PBMC) are isolated using the commercially available Pan T cell Isolation Kit, human.

4) Pan T cells are cultivated in TexMACSTM Medium supplemented with IL-7 and IL-15. Cells are activated with T Cell TransAct.

5) T cells are transduced with fluorescence protein construct using a lentiviral vector.

6) CAP treated peptides are directly applied to naïve T cell culture or to dendritic cell culture for 3 to 7 days

7) Expansion of T cells will be done in TexMACS Medium supplemented with IL-7 and IL-15.

8) Cells are split every 2-3 days

9) T cell culture are wash and cytokines removed.

10) A subset of T cells will be co-cultured with target cells for 48 h.

11) Killing of target cells will be analyzed.

12) Upon successful killing of target cells, T cells will be isolated and stored until administer to patient.

The peptide sequences with 15 amino acids flanking both sides of the epitope of the peptides are as follows (SEQ ID NOS: 9-11, respectively, in order of appearance.):

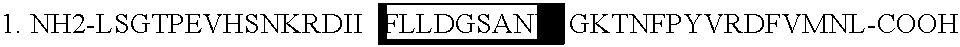

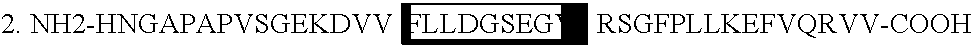

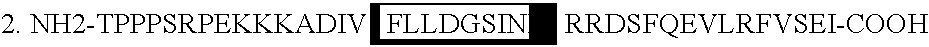

These peptides could be chemically modified, such as Polyethylene glycol (PEG) modification (PEGylation) and two or more amino acid bases could be substituted with other amino acids within the peptide sequences.

In another embodiment, the present invention uses cold atmospheric plasma-treated cancer epitope peptides within the KRAS protein as a PDAC vaccine. The cold atmospheric plasma treatment will increase the immunogenicity of the KRAS protein, making it a more effective target for cancer immunotherapy.

The vaccine is composed of cancer epitope peptides within the KRAS protein that have been treated with cold atmospheric plasma. The cold atmospheric plasma treatment will increase the immunogenicity of the peptides, making them more effective at eliciting an immune response against PDAC cells. The vaccine will be administered to patients with PDAC as a stand-alone vaccine and/an adjuvant treatment before or following surgical resection of the tumor. The cold atmospheric plasma-treated vaccine will be sprayed onto the surgical margins to eradicate any residual cancer cells and prevent tumor recurrence.

The vaccine for the treatment of pancreatic ductal adenocarcinoma (PDAC) comprises cancer epitope peptides within the Kirsten Rat Sarcoma 2 Viral Oncogene Homologue (KRAS) protein that have been treated with cold atmospheric plasma. This includes the following peptides:

Peptide 1. Mutant p.G12D Sequence:
(SEQ ID NO: 4)
NH2-MTEYKLVVVGADGVGKSALTIQLI-COOH;
Peptide 2. Mutant p.G12V Sequence:
(SEQ ID NO: 5)
NH2-MTEYKLVVVGAVGVGKSALTIQLI-COOH;
Peptide 3. Mutant p.G12R Sequence:
(SEQ ID NO: 6)
NH2-MTEYKLVVVGARGVGKSALTIQLI-COOH;
Peptide 4. Mutant p.G12C Sequence:
(SEQ ID NO: 7)
NH2-MTEYKLVVVGACGVGKSALTIQLI-COOH;
and
Peptide 5. Mutant p.G12S Sequence:
(SEQ ID NO: 8)
NH2-MTEYKLVVVGASGVGKSALTIQLI-COOH.

The cold atmospheric plasma treatment increases the immunogenicity of the cancer epitope peptides, making them more effective at eliciting an immune response against PDAC cells.

In yet another embodiment, the present invention is a method of treating PDAC comprising administering the vaccine to a patient intravenously.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto and their equivalents. The entirety of each of the aforementioned documents is incorporated by reference herein.

1. A method to oxidize pan-cancer epitopes of COL6A3 protein (peptide sequences FLLDGSANV (SEQ ID NO: 1), FLLDGSEGV (SEQ ID NO: 2) and FLLDGSINF (SEQ ID NO: 3)) by cold atmospheric plasma treatment for developing solid tumor cancer vaccines, comprising:
synthesizing and solubizing peptides FLLDGSANV (SEQ ID NO: 1), FLLDGSEGV (SEQ ID NO: 2) and FLLDGSINF (SEQ ID NO: 3) in an appropriate solvent such as PBS or DMSO;
treating the synthesized peptides with cold atmospheric plasma (CAP);
isolating Pan T cells from peripheral blood mononuclear cells (PBMC);
cultivating Pan T cells in TexMACSTM Medium supplemented with IL-7 and IL-15;
activating the Pan T cells with T Cell TransAct;
transducing the Pan T cells with fluorescence protein construct using a lentiviral vector;
directly applying CAP treated peptides to naïve T cell culture or to dendritic cell culture for 3 to 7 days;
expanding T cells in TexMACS Medium supplemented with IL-7 and IL-15, T cells are split every 2-3 days;
washing T cell cultures and remove cytokines.
co-culturing a subset of T cells with target cells for 48 h;
analyzing killing of target cells; and
upon successful killing of target cells, isolating and storing T cells until administered to patient.

2. (canceled)

3. The method according to claim 1, wherein the isolating of Pan T cells from peripheral blood mononuclear cells (PBMC) is performed using a commercially available Pan T cell Isolation Kit, human.

4. The method according to claim 1, wherein the step of treating the synthesized peptides with cold atmospheric plasma comprises treating the synthesized peptides with cold atmospheric plasma at a power ranging from 1 v to 120 v, and time from 1 second to 7 minutes with 1 to 3 liters per minute of He.

5. (canceled)

6. (canceled)
